(19) United States
(12) Patent Application Publication
(10) Pub. No.: US 2025/0213842 A1
(43) Pub. Date: Jul. 3, 2025

(54) APERTURE SEAL VALVE FOR HEMOSTASIS

(71) Applicant: EDWARDS LIFESCIENCES CORPORATION, Irvine, CA (US)

(72) Inventors: Nithya Sridhar, Irvine, CA (US); Joshua Thomas Derr, Irvine, CA (US); Maria G. Verastegui Jaimez, Aliso Viejo, CA (US)

(73) Assignee: EDWARDS LIFESCIENCES CORPORATION, Irvine, CA (US)

(21) Appl. No.: 19/085,861

(22) Filed: Mar. 20, 2025

Related U.S. Application Data
(63) Continuation of application No. PCT/US2023/074992, filed on Sep. 25, 2023.
(60) Provisional application No. 63/377,315, filed on Sep. 27, 2022.

(51) Int. Cl. A61M 39/06; A61M 25/06

(52) U.S. Cl. CPC A61M 39/06; A61M 25/0662; A61M 2025/0681; A61M 2039/068

(57) ABSTRACT

An aperture valve for maintaining hemostasis extends from a proximal end to a distal end along a primary axis. The aperture valve includes a proximal ring situated at the proximal end and formed of rigid material, a distal ring situated at the distal end and formed of rigid material, a flexible cylinder surrounding the primary axis, attached to and axially overlapping with the proximal ring and the distal ring, and a locking mechanism selectively anchoring the proximal ring to the distal ring. The flexible cylinder is irisable by rotation of the proximal ring relative to the distal ring. The locking mechanism is engageable to retain the flexible cylinder in an irised state by locking the proximal ring to the distal ring while the proximal ring is rotated relative to the distal ring.

### CROSS-REFERENCE TO RELATED APPLICATION(S)

This application is a continuation of International Application No. PCT/US2023/074992, filed Sep. 25, 2023, which claims the benefit of U.S. Provisional Application No. 63/377,315, filed Sep. 27, 2022, the disclosures of which are hereby incorporated by reference in their entireties.

### BACKGROUND

The present disclosure relates generally to medical equipment, and more particularly to valves configured to maintain hemostasis.

Medical devices inserted into the vasculature of a patient must be sealed to prevent fluid (e.g. blood) leakage. In some instances, fluid seals can be provided through stacked sealing elements. Among these sealing elements are hemostasis (haemostasis) valves configured to stop the flow of blood while a medical device is inserted into a vein or artery of the patient. Such hemostasis valves form a fluid seal between the patient's body and medical instrumentation.

When inserted at some locations, e.g. for cardiac catheterization via the jugular vein, medical devices can make sharp turns near an insertion device (i.e. access sheath), within patient vasculature. Such sharp turns can stress or deform many types of hemostatic seals, permitting leakage. Hemostatic seals capable of accommodating sharp turns without leakage are desirable to maintain hemostasis when the medical devices are installed at such locations.

### SUMMARY

In one example, the disclosure presents an aperture valve for maintaining hemostasis. This aperture valve extends from a proximal end to a distal end along a primary axis. The aperture valve includes a proximal ring situated at the proximal end and formed of rigid material, a distal ring situated at the distal end and formed of rigid material, a flexible cylinder surrounding the primary axis, attached to and axially overlapping with the proximal ring and the distal ring, and a locking mechanism selectively anchoring the proximal ring to the distal ring. The flexible cylinder is irisable by rotation of the proximal ring relative to the distal ring. The locking mechanism is engageable to retain the flexible cylinder in an irised state by locking the proximal ring to the distal ring while the proximal ring is rotated relative to the distal ring.

In another example, this disclosure presents a guide sheath assembly for maintaining hemostasis during vascular insertion of an insertable medical device into a patient. The guide sheath assembly extends along a central axis from a proximal end to a distal end closest to the patient, and includes a sheath shaft, a seal stack, and a handle providing an exterior housing retaining and at least partially coaxially surrounding the sheath shaft and the seal stack. The sheath shaft is coaxial with the central axis and is disposed at the proximal end to axially receive the insertable medical device. The seal stack forms a hemostatic seal distal from the sheath shaft, and includes a pre-insertion valve and an irising aperture valve. The pre-insertion valve is configured to form a hemostatic seal when the insertable medical device is not inserted through the guide sheath assembly. The irising aperture valve is coaxial with the central axis and is located distally relative to the pre-insertion valve. The irising aperture valve includes a rigid proximal ring, a rigid distal ring, and a flexible cylinder connecting the rigid proximal ring to the rigid distal ring. The rigid distal ring is axially translatable and angularly rotatable relative to the rigid proximal ring. The flexible cylinder is irisable with rotation of the rigid distal ring relative to the rigid proximal ring.

In yet another example, this disclosure presents a method of forming a hemostatic seal about a medical device at a vascular insertion site. According to this method, the medical device is extended through a sheath shaft to the insertion site, along a central axis. An aperture seal valve is interposed between the sheath shaft and the insertion site. This aperture seal valve includes a proximal ring, a distal ring, a flexible cylinder connecting the proximal ring to the distal ring, a plurality of teeth, and a locking mechanism. The distal ring is axially translatable and angularly rotatable relative to the proximal ring to iris the flexible cylinder, and the plurality of teeth are disposed at an intersection of the flexible cylinder and at least one of the proximal and distal rings. A fluid seal about the medical device is formed by rotating the distal ring relative to the proximal ring, thereby irising the flexible cylinder. This fluid seal is retained about the medical device by engaging the locking mechanism to lock the proximal ring relative to the distal ring.

### BRIEF DESCRIPTION OF THE DRAWINGS

FIG. 1A is a simplified perspective view of a guide sheath assembly including an aperture seal valve.

FIG. 1B is a cross-sectional view of the guide sheath assembly of FIG. 1A.

FIG. 2A is a perspective view of an aperture seal valve as presented in FIGS. 1A and 1B.

FIG. 2B is side view of the aperture seal valve of FIG. 2A.

FIG. 2C is an axial view of the aperture seal valve of FIG. 2A.

FIG. 3A is an axial view of an aperture seal valve in an open state.

FIG. 3B is an axial view of an aperture seal valve in a partially closed state.

FIG. 3C is an axial view of an aperture seal valve in a partially open state.

FIG. 3D is an axial view of an aperture seal valve in a closed state.

FIG. 4A is a simplified side view of an aperture seal valve in a twisted open state.

FIG. 4B is a simplified side view of an aperture seal valve in a twisted closed state.

FIG. 5A is a perspective view of one example of an aperture seal valve with a flat slot interlock, in a closed state.

FIG. 5B is a perspective view of another example of an aperture seal valve with a curved slot interlock, in an open state.

FIGS. 6A-6E are simplified perspective views of the aperture seal valve of FIG. 5B through a succession of locking stages.

FIG. 7 is a perspective view of the aperture seal valve of FIG. 5B in an open state with an action line illustrating a locking path of an interlocking feature.

### DETAILED DESCRIPTION

This disclosure relates to a hemostasis maintenance device including an irising aperture seal valve. This aperture seal valve can be twisted, telescoped, and locked to constrict a flexible passage to form a fluid seal about medical instrumentation, thereby preventing or reducing leakage of blood and bodily fluids during an off-axis insertion of such medical instrumentation into vasculature of the patient. The twisting of the seal valve constricts a geometric aperture, the telescoping of the seal compresses this aperture, and locking the seal maintains the constriction and compression of the aperture.

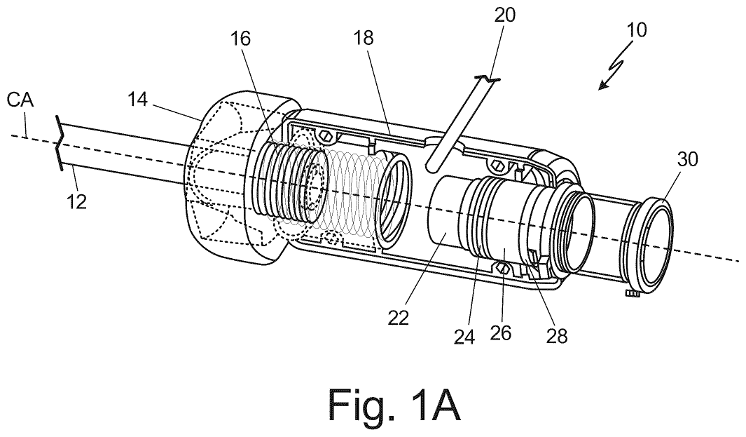

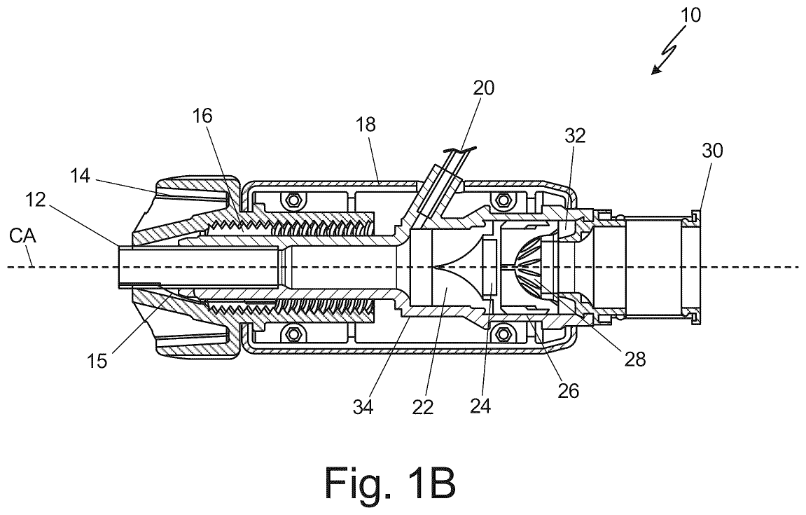

FIGS. 1A-1B illustrate an example of guide sheath assembly 10 with aperture valve 30, FIGS. 1A-1B will be discussed together. FIG. 1A provides a simplified perspective view, while FIG. 1B provides a cross-sectional view. As illustrated in FIGS. 1A & 1B, guide sheath assembly 10 includes aperture valve 30, sheath shaft 12, knob 14, shaft line 15, nut 16, handle 18, flush tube 20, duckbill valve 22, disc valve 24, spacer 26, cross slit valve 28, connecting cap 32, hub seal 34, and central axis CA. Shaft line 15, connecting cap 32, and hub seal 34 are not illustrated in FIG. 1A; see FIG. 1B.

Guide sheath assembly 10 is a stack or assembly of components which together function to allow insertion of a medical device into the vasculature of a patient while maintaining hemostasis. Prevention of blood leakage from an insertion site is necessary to limit any drop in blood pressure, reduce reliance on blood transfusion, and generally safeguard patient health. The medical device inserted can include a catheter, sensor, guide wire, or any other medical device small enough to fit through sheath shaft 12. Sheath shaft 12 can for example be a 20F sheath shaft, or more generally any other size of sheath shaft known to those of skill in the art as being insertable into the vasculature of the patient. Although guide sheath assembly 10 and aperture valve 30 are described generically herein, analogous but dimensionally distinct versions of these devices can used for specific types and dimensions of insertable medical equipment without departure from the spirit of the present disclosure. In some applications, multiple such distinct but analogous devices can be used to form hemostatic seals for different apparatus and/or at different applications.

Sheath shaft 12 of guide sheath assembly 10 defines an access path by which a medical device is inserted into patient vasculature. Sheath shaft 12 is located at a first end of guide sheath assembly 10 nearest the patient. Sheath shaft 12 can, for example, be inserted into the vasculature of the patient through the use of an introducer configured to puncture the patient's skin. Alternatively, a physician can create a small incision into the skin of the patient allowing access to the vasculature. Sheath shaft 12 can be formed of a flexible plastic, silicone, or any other biocompatible material known to those of skill in the art. Central axis CA is defined by an axial center of sheath shaft 12. Central axis CA has a distal end nearest to the patient and a proximal end opposite the distal end.

Coaxial with sheath shaft 12 are knob 14 and nut 16. In the illustrated example, knob 14 engages with nut 16 via threading. Nut 16 is situated radially between sheath shaft 12 and knob 14. Knob 14 can be the radially outermost component of guide sheath assembly 10 near the distal end of central axis CA. Shaft line 15 connects through nut 16 along an axis parallel to central axis CA. Shaft line 15 runs from nut 16 to sheath shaft 12. Shaft line 15 runs along a radially inner side of sheath shaft 12. When knob 14 and nut 16 are in a first state, shaft line 15 is loose, allowing sheath shaft 12 to flex. When knob 14 and nut 16 are in a second state, a length of shaft line 15 is wrapped around a radially outer side of sheath shaft 12 and shaft line 15 becomes taught, thereby causing sheath shaft 12 to become rigid. Knob 14 and nut 16 are rigid structural elements that can, for example, be formed of plastic, metal, or any other material capable of holding its shape under compressive forces.

Handle 18 is a structural housing that surrounds, contains, and protects all of the components of guide sheath assembly 10 except sheath shaft 12 and knob 14. Further, handle 18 provides a grip that can be held by a human operator. Handle 18 can be shaped as a hollow cylinder with end caps. Handle 18 can be built of any suitable rigid material.

Flush tube 20 can be a hollow cylinder that connects to sheath shaft 12. In the example shown in FIG. 1A, flush tube 20 extends radially outwards from central axis CA of sheath shaft 12 through a circular opening in handle 18. When air must be removed from guide sheath assembly 10 prior to a procedure, the operator can flush saline through flush tube 20 to remove air from guide sheath assembly 10. Flush tube 20 can be formed of a flexible plastic, silicone, or any other material known to those of skill in the art as being biocompatible.

Duckbill valve 22, disk valve 24, cross slit valve 28, and aperture valve 30 work together to maintain hemostasis. As shown in the example of FIG. 1A, duckbill valve 22 is closest to the distal end of guide sheath assembly, while aperture valve 30 is at the proximal end of guide sheath assembly 10. As shown in the example of FIG. 1B, from the distal end to the proximal end the valves are arranged such that duckbill valve 22 connects to disk valve 24 which connects to cross slit valve 28 which connects to aperture valve 30. As shown in the example of FIG. 1B, duckbill valve 22, disk valve 24, cross slit valve 28, and aperture valve 30 are arranged co-axially with central axis CA. Each valve contributes to optimal sealing under different conditions or use cases. In specific illustrative examples, duckbill valve 22 provides a fluid seal when there is nothing inserted through the device (i.e. acting as a pre-insertion seal), while disc valve 24 and cross slit valve 28 provide efficient fluid seals suited specifically to a catheter or a guidewire, respectively, once inserted through the device. Aperture valve 30 supplements the seals provided by the three other valves, especially when the inserted objected is not coaxial with the central axis CA. Duckbill valve 22, disk valve 24, cross slit valve 28, and flexible portions of aperture valve 30 (e.g. flexible cylinder 40) can be formed of silicone, polyvinylchloride, polyethylene, polypropylene, polytetrafluoroethylene, polymethylmethacrylate, trimethyl carbonate, or any combination thereof or other material known to those of skill in the art as being biocompatible and able to reversibly deform. Flexible cylinder 40, specifically, is formed of a reversibly elastically deformable material.

As can best be seen in FIG. 1B, spacer 26 radially surrounds cross slit valve 28. Spacer 26 axially extends from a distal end of cross slit valve 28 to near a proximal end of cross slit valve 28. Spacer 26 ensures that cross slit valve 28 is not over compressed and further ensures that a sufficient gap is present between disk valve 24 and cross slit valve 28 such that both disk valve 24 and cross slit valve 28 are able to function properly. Spacer 26 can be constructed of any rigid material capable of holding its shape under expected compressive forces.

Connecting cap 32 is disposed at a proximal end of handle 18, opposite knob 14. Connecting cap 32 provides access into handle 18 for the medical devices to be inserted. Connecting cap 32 is coaxial with sheath shaft 12, and interfaces with aperture valve 30 to reversibly lock aperture valve 30 in alignment with other sealing elements of sheath assembly 10. Connecting cap 32 further functions to provide compression to duckbill valve 22, disk valve 24, and cross slit valve 28. Hub 34 radially surrounds and houses duckbill valve 22, disk valve 24, spacer 26, and cross slit valve 28, and is situated within handle 18. Hub 34 connects to sheath shaft 12, flush tube 20, and connecting cap 32, thereby connecting all of the components within guide sheath assembly 10. Hub 34 can be shaped as an elongated cylinder with a first diameter near the distal end which flares outward to a second diameter towards the proximal end to accommodate duckbill valve 22, disk valve 24, spacer 26, and cross slit valve 28. Connecting cap 32 and hub 34 can be constructed of any rigid material capable of holding its shape under expected forces.

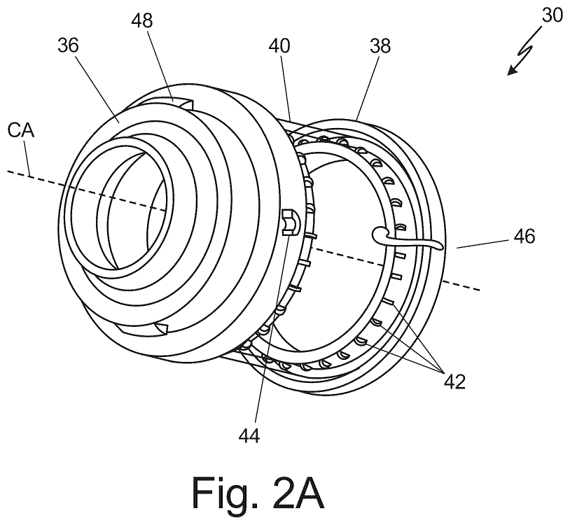

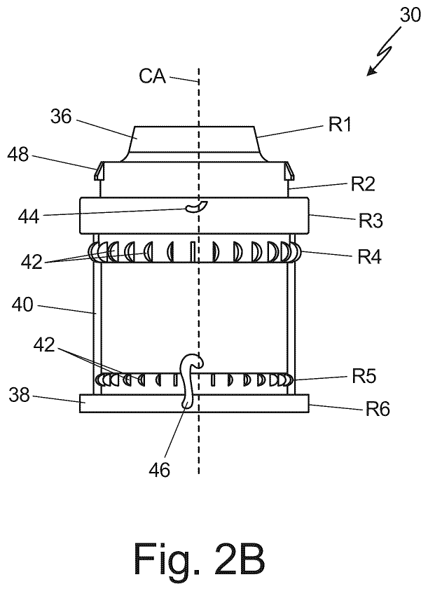

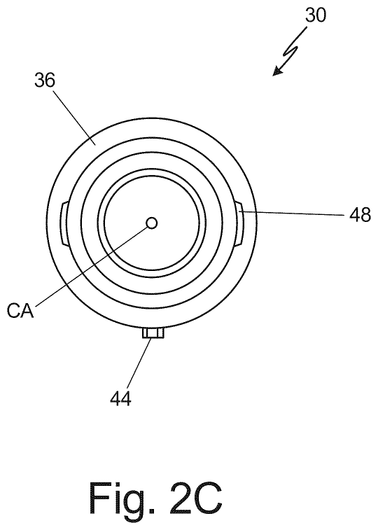

FIGS. 2A-2C disclose an example of aperture valve 30 and will be discussed together. FIG. 2A is a simplified perspective view of aperture valve 30. FIG. 2B is an side view of aperture valve 30 of FIG. 2A. FIG. 2C is an end-on axial view of aperture valve 30 of FIG. 2A. FIGS. 2A-2C collectively illustrate proximal ring 36, distal ring 38 (not shown in FIG. 2C), flexible cylinder 40, teeth 42, curved receptacle 44, curved protrusion 46, distal cap lock 48, and central axis CA. Proximal ring 36 includes first ring section R1, second ring section R2, third ring section R3, and fourth ring section R4. Distal ring 38 includes fifth ring section R5 and sixth ring section R6. As used throughout, the terms “distal” and “proximal” refer to relative proximity to a physician or other operator, i.e. with the distal end of the device being closest to the patient once installed.

As discussed above with respect to FIG. 1, aperture valve 30 is a valve which improves hemostasis provided by guide sheath assembly 10. If the device to be inserted via guide sheath assembly 10 to sheath shaft 12 is off of central axis CA or is pulled off central axis CA due to working limitations imposed by the orientation of the catheter setup, seals of duck bill valve 22, disc valve 24, and cross slit valve 28 can be pulled open, thereby losing hemostasis. Aperture valve 30 allows hemostasis to be maintained when the device inserted is not coaxial with central axis CA. As described in detail below, aperture valve 30 best maintains hemostasis when aperture valve 30 is twisted, telescoped, and locked.

Proximal ring 36 and distal ring 38 are located at opposite first and second ends, respectively, of aperture valve 30. Distal and proximal rings 36, 38 are hollow cylinders with varying outer diameters. In the example shown in FIGS. 2A-2C, proximal ring 36 is integrally formed of four ring sections of differing outer diameters, while distal ring 38 is integrally formed of two ring sections of different diameters. Flexible cylinder 40 extends between and is attached to both proximal ring 36 and distal ring 38 in a substantially fluid impermeable connection. Flexible cylinder 40 can, in some examples, be formed of a pliable polymer material.

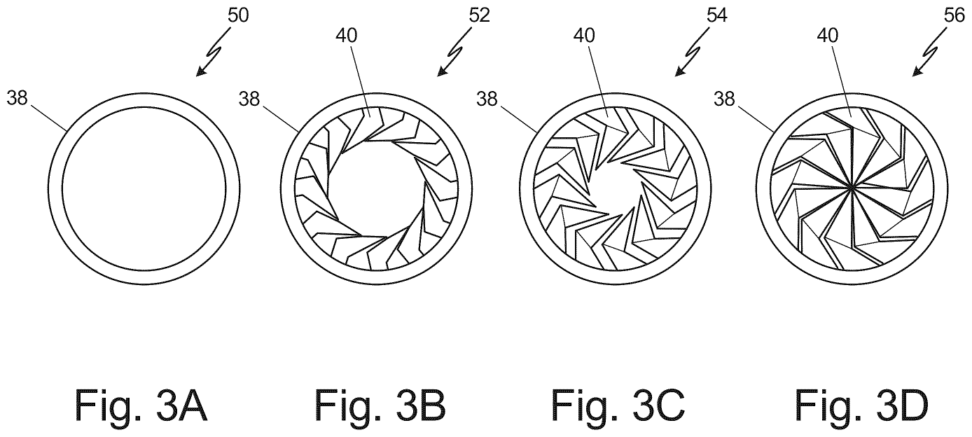

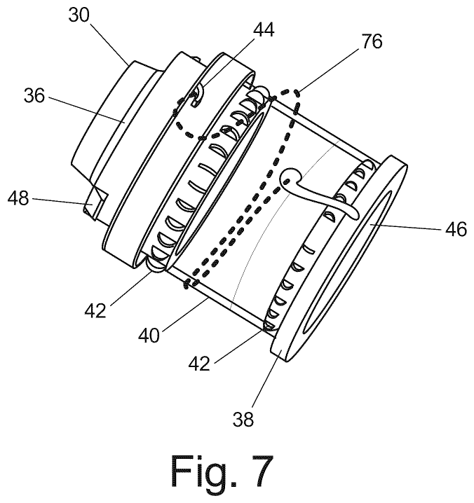

Generally, proximal ring 36 and distal ring 38 each include a ring section (R4 and R5, respectively) that axially overlaps with and attaches to flexible cylinder 40, and an adjacent ring section (R3 and R6, respectively) that abuts but does not axially overlap flexible cylinder 40. At least one and preferably both of ring sections R4 and R5 include radially-extending, circumferentially distributed teeth 52 within the overlap of ring sections R4/R5 with flexible cylinder 40. Teeth 52 engage flexible cylinder 40, improving retention of flexible cylinder 40 and promoting folding of flexible cylinder 40 into an irised shape, i.e. with overlapping folds, when proximal ring 36 is twisted relative to distal ring 38, as described further with respect to FIGS. 3A-3D. Ring sections R3 and R6 extend radially outward of ring sections R4 and R5, and of flexible cylinder 40, and engage each other via a locking mechanism (e.g. through the interface of curved receptacle 44 with curved protrusion 46; see FIG. 7 and accompanying description, below) to retain proximal ring 36 in a locked position relative to distal ring 38.

According to the example illustrated in FIGS. 2A and 2B, proximal ring 36 is formed of four ring sections. In addition to ring sections R3 and R4, discussed above, proximal ring 36 includes ring sections R1 and R2 at the most distal end of aperture valve 30 with respect to a patient insertion point. In the illustrated example, ring section R1 has the smallest diameter, and tapers frustoconically to a narrowest diameter at proximal end of aperture valve 30 to accommodate positioning within guide sheath assembly 10. Ring section R2 is situated immediately between ring section R1 and ring section R3 and includes distal cap lock 48. Distal cap lock 48 is formed onto a radially outer surface of ring section R2 and enables aperture valve 30 to removably interlock into hub seal 34. Distal cap lock 48 can be a wedge, flange, or circumferential projection that extends radially outwards from ring section R2 to engage hub seal 34.

In some embodiments of aperture valve 30, any or all of ring sections R1-R4 can be tapered. In a further alternative example, ring section R1 need not be tapered. In the example shown in FIGS. 2A-2C, transitions between ring sections R1-R4 are filleted to reduce stress concentration points. In the example shown in FIGS. 2A-2C, ring sections R1-R4 have a common inner diameter defining a continuous cylindrical inner surface. Ring sections R1-R4 together form proximal ring 36, which reversibly connects to connecting cap 32.

As noted above, flexible cylinder 40 extends between proximal ring 36 and distal ring 38. Flexible cylinder 40 is deformed during twisting and telescoping of the aperture valve 30 to create a hemostatic seal about the inserted medical device. In the example of FIGS. 2A-2C, an outer diameter of flexible cylinder 40 is larger than the outer diameter ring section R5 but smaller than the outer diameter of ring section R6. Further, the outer diameter of flexible cylinder 40 is larger than the outer diameter of ring section R4 but smaller than the outer diameter of ring section R3. The inner diameter of flexible cylinder 40 can, for example, be substantially equal to the outer diameter of ring sections R4 and R5. The inner diameter of flexible cylinder 40 defines a flow path that runs between ring sections R4 and R5, and cooperates with internal diameters of distal and distal rings 36 and 38 to define a fluid channel extending from the first end to the second end of aperture valve 30. Ring sections R4 and R5 have radial thicknesses less than corresponding thicknesses of ring sections R3 and R6, respectively. In the illustrated example, flexible cylinder 40 fully overlaps ring sections R4 and R5, but in other examples flexible cylinder 40 can overlap only a portion of ring sections R4 and R5.

Flexible cylinder 40 can be secured to proximal ring 36 and distal ring 38 by melting and reforming portions of flexible cylinder 40 that overlap with proximal ring 36 and distal ring 38, causing teeth 42 to melt into and bond with flexible cylinder 40. Although FIGS. 2A and 2B illustrate teeth 42 extending radially outward from ring sections R4 and R5, alternative examples of aperture valve 30 can include teeth 42 that project radially inward, to engage and bond with flexible cylinder 40. In further examples, flexible cylinder 40 can additionally or alternatively be glued to proximal ring 36 and distal ring 38 with adhesives.

Flexible cylinder 40 is shown disposed radially outward of and coaxially surrounding ring sections R4 and R5, and directly abutting a radial step in the outer diameters of distal ring 36 and distal ring 38 between ring sections R3/R4 and R5/R6, respectively. In other examples, however, flexible cylinder 40 can be disposed radially inboard of ring sections R4 and R5, in which configurations flexible cylinder 40 can, for example, abut a radial step in the corresponding inner diameter of distal ring 36 and distal ring 38 between ring sections R3/R4 and R5/R6, respectively. In these configurations, teeth 42 extend radially inward from ring sections R4 and R5 to engage flexible cylinder 40.

As noted above, proximal ring 36 anchors curved receptacle 44, a retention slot or groove formed onto a radially outer side of ring section R3 of proximal ring 36. Distal ring 38 supports curved protrusion 46, a complementary latching mechanism extending axially from a radially outer side of ring section R5. Curved protrusion 46 extends axially from distal ring 38 toward proximal ring 36, radially outward of flexible cylinder 40. When proximal ring 36 is locked to distal ring 38, curved receptacle 44 latches into or otherwise interlocks with curved protrusion 46 to keep proximal ring 36 and distal ring 38 adjacent to each other.

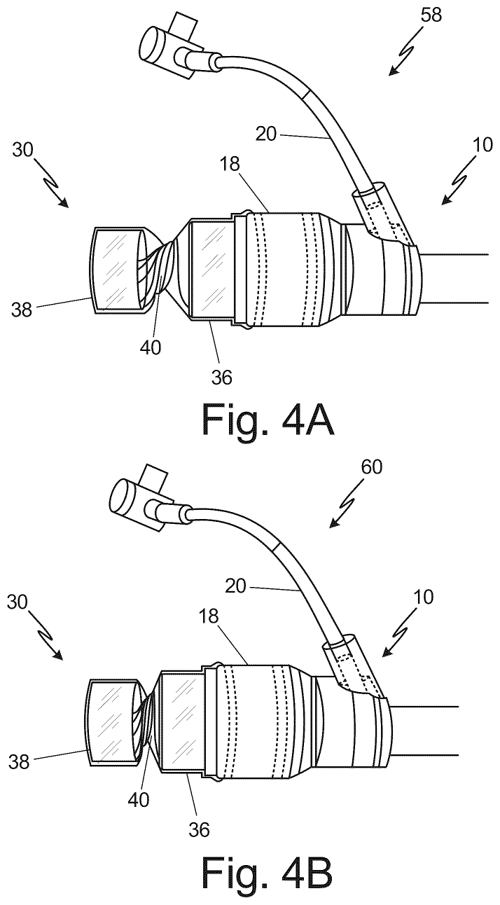

FIGS. 3A-3D illustrate aperture valve 30 in successive stages of closure and will be discussed together. FIG. 3A provides an axial view of aperture valve 30 in open iris state 50, while FIG. 4D provides an axial view of aperture valve 30 in closed iris state 56. FIGS. 3B and 3C provide axial views of aperture valve 30 in intermediate mostly open and mostly closed iris states 52 and 54, respectively. Iris states 50-56 represent degrees of irising of flexible cylinder 40 accomplished by rotation and axial displacement of distal ring 38 relative to proximal ring 36. In all iris states, flexible cylinder 40 can remain substantially coaxial with proximal ring 36 and distal ring 38.

In open iris state 50, flexible cylinder 40 remains undeformed (i.e. cylindrical) and no geometric aperture is yet formed, leaving aperture valve 30 in its most open state. In mostly open iris state 52, distal ring 38 has been twisted about central axis CA relative proximal ring 36. In mostly open iris state 52, flexible cylinder 40 is deformed as a result of the rotational twisting, resulting in a slight reduction to an inner diameter of flexible cylinder 40—and consequently to aperture valve 30 as a whole. In mostly closed iris state 54, distal ring 38 is further twisted about central axis CA relative proximal ring 36, further deforming flexible cylinder 40 and consequently further reducing the inner diameter of flexible cylinder 40. In closed iris state 56, distal ring 38 is further twisted about central axis CA relative proximal ring 36 deforming flexible cylinder 40 so substantially as to reduce the inner diameter of flexible cylinder 40 to approximately zero and fully close the iris of aperture valve 30. In closed iris state 56, flexible cylinder 40 completely occludes the flow path. Although closed iris state 56 is illustrated for explanatory purposes, some embodiments of aperture valve 30 may not support fully closing the iris of aperture valve 30. More specifically, the degree of irising necessary to fully support a hemostatic seal will depend on the diameter of instrumentation or other medical apparatus inserted through aperture valve 30. In general, aperture valve 30 is sufficiently “closed” if rotation of distal ring 38 relative to proximal ring 36 deforms flexible cylinder 40 into close enough contact with the inserted apparatus to maintain a fluid seal despite movement, jostling, etc. of the apparatus. In this state, the inner diameter of flexible cylinder 40 is less than corresponding inner diameters of proximal ring 36 and distal ring 38.

All deformations of flexible cylinder 40 are elastic deformations generating a restoring (i.e. spring) force in the opposite direction of the rotation due to elasticity of flexible cylinder 40. Each iris stage can be reversed by twisting distal ring 38 about central axis CA relative proximal ring 36 in the opposite rotational direction. In the illustrative example shown in FIGS. 3A-3D, all rotations of distal ring 38 are counterclockwise, and are consequently reversible via clockwise rotation. As flexible cylinder 40 is deformed inwards, an axial distance between proximal ring 36 and distal ring 38 is reduced due the limited distance flexible cylinder 40 is able to stretch. Telescoping of aperture valve 30 will be discussed further with respect to FIGS. 4A-4B.

FIGS. 4A-4B are exaggerated perspective views illustrating guide sheath assembly 10 with aperture valve 30. Guide sheath assembly 10 further includes handle 18 and flush tube 20. Aperture valve 30 includes proximal ring 36, distal ring 38, and flexible cylinder 40. FIGS. 4A-4B depict twisted open 58 and twisted closed 60 states, respectively, and will be discussed together. FIG. 4A is a side view of aperture valve 30 in a twisted open 58 state. FIG. 4B is a side view of aperture valve 30 in a twisted closed 60 state. Twisted open state 58 represents a state of guide sheath assembly 10 wherein flexible cylinder 40 is partially twisted. Twisted closed state 60 represents a state of guide sheath assembly 10 wherein flexible cylinder 40 is twisted further (relative to twisted open state 58) to form a fluid seal about a substantially coaxial insert (not shown).

Handle 18, flush tube 20, aperture valve 30, proximal ring 36, distal ring 38, and flexible cylinder 40 are substantially as described above with respect to 1A-2C. In twisted open state 58, aperture valve 30 has been twisted, e.g. into mostly open iris state 52 such that the inner diameter of flexible cylinder 40 is somewhat reduced, thereby also reducing an axial distance between proximal ring 36 and distal ring 38. In twisted open stage 58, hemostasis has not yet been secured, and distal ring 38 of aperture valve 30 has not been telescoped to and locked to proximal ring 36. In twisted closed stage 60, distal ring 38 of aperture valve 30 has been rotated further and axially telescoped along central axis CA towards proximal ring 36, and distal ring 38 has been locked onto proximal ring 36. Proximal ring 36 and distal ring 38 can be locked together through latching engagement of curved protrusion 46 onto/into curved receptacle 44, or any other example of an interlocking mechanism. In twisted closed stage 60, the aperture formed by closed iris state 56 is axially compressed about the medical insert, thereby forming a hemostatic seal.

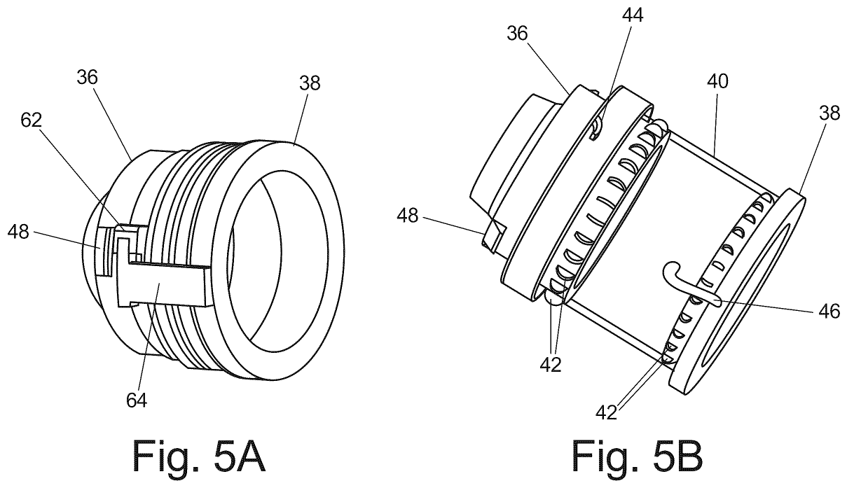

FIGS. 5A-5B illustrate examples of locking mechanisms for aperture valve 30. FIG. 5A is a perspective view of aperture valve 30 with a flat slot interlock. FIG. 5A depicts proximal ring 36, distal ring 38, distal cap lock 48, flat receptacle 62, and flat protrusion 64. FIG. 5B is a perspective view of a radial seal valve with a curved slot interlock as introduced previous with reference to FIG. 2B. FIG. 5B depicts proximal ring 36, distal ring 38, flexible cylinder 40, teeth 42, curved receptacle 44, curved protrusion 46, and distal cap lock 48.

The locking mechanisms shown in FIGS. 5A and 5B enable proximal ring 36 to be secured to distal ring 38 such that twisted closed stage 60 (as shown in FIG. 4B) can be maintained without continuous human intervention. As discussed above with respect to FIG. 4B, maintaining twisted closed stage 60 allows for hemostasis to be maintained. As illustrated in FIGS. 5A and 5B, the locking mechanism of FIG. 5A comprises flat receptacle 62 and flat protrusion 64. Similarly to curved receptacle 44, flat receptacle 62 is attached to a radially outward side of third ring section R3. Similarly to curved protrusion 46, flat protrusion 64 is attached to the radially outward side of fifth ring R5. Flat protrusion 64 extends axially along a radially outer surface of flexible cylinder 40 towards proximal ring 36. Flat protrusion 64 is a flange or extension that extends radially from a portion of flat protrusion 64 nearest to proximal ring 36. When distal ring 38 is adjacent proximal ring 36, flat protrusion 64 interlocks with flat receptacle 62. When deformed, the counterrotational bias of flexible cylinder 40 provides a rotational restoring (spring) force that keeps flat protrusion 64 interlocked with flat receptacle 62.

The locking mechanism shown in FIG. 5B operates substantially as described above in reference to FIGS. 2A-2C. As described previously, the aforementioned counterrotational restoring force provided by elasticity of flexible cylinder 40 retains a curved portion of curved protrusion 46 in curved receptacle 44. In further examples, locking mechanisms can include ratchetable fasteners securable at multiple angular positions. Although the present figures provide two example of locking mechanisms including curved or flat mating receptacles and protrusions, other examples can include multiple circumferentially distributed receptacles, troughs, or threadings disposed to lockingly engage with one or more complementary heads or protrusions. More specifically, multiple receptacles (e.g. grooves, teeth, etc.) disposed across an angular range of the circumference of proximal ring 36 or distal ring 38—including up to 100% of the circumference of either ring—can allow distal and proximal rings 38, 36 to be locked relative to each other at a range of different angular offsets. This flexibility permits in locking angle produces a corresponding flexibility in aperture irising of flexible cylinder 40, allowing aperture valve 30 to accommodate catheter insertions of different dimensions and materials.

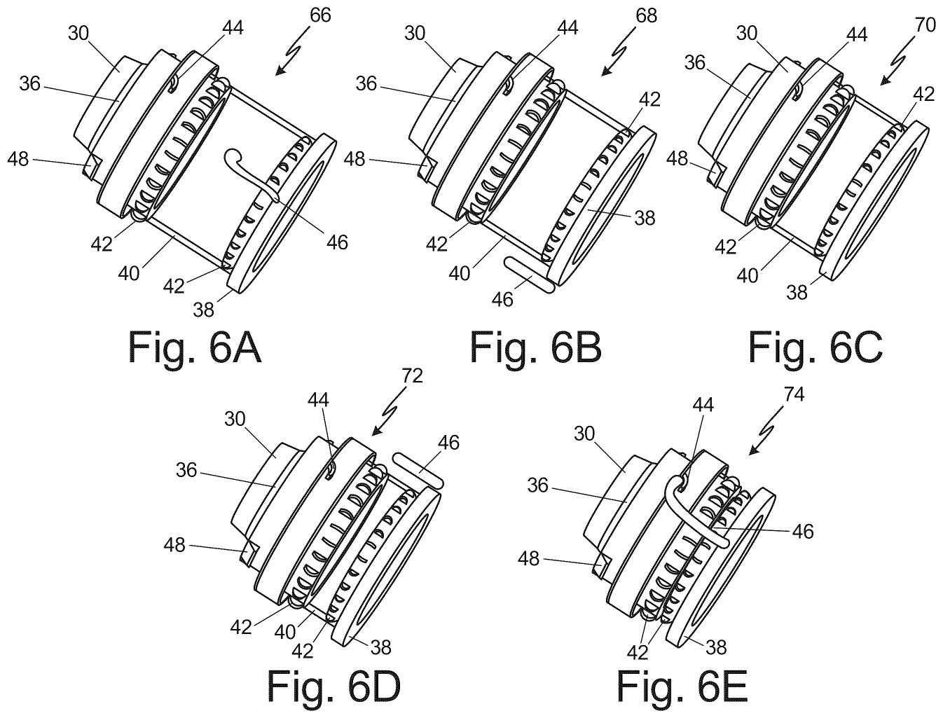

FIGS. 6A-7 discloses aperture valve 30 in multiple stages of closure and locking path 76 of such a closure and will be discussed together. FIGS. 6A-7 are exaggerated for demonstrative purposes, and are not necessarily drawn to scale. FIG. 6A-6D are a perspective views of first, second, third, fourth, and fifth stages 66-74, respectively, of closing aperture valve 30. FIG. 7 is a perspective view of aperture valve 30 with a coiling action line illustrating locking path 76, an exaggerated path taking aperture valve 30 from first stage 66 to fifth stage 74. FIGS. 6A-7 illustrate aperture valve 30, proximal ring 36, distal ring 38, flexible cylinder 40 (not shown in FIG. 6E), teeth 42, curved receptacle 44, curved protrusion 46 (not shown in FIG. 6C), and distal cap lock 48.

Aperture valve 30, proximal ring 36, distal ring 38, flexible cylinder 40, teeth 42, curved receptacle 44, curved protrusion 46, and distal cap lock 48 are substantially as described above. When distal ring 38 is twisted counterclockwise relative to proximal ring 36, the length of flexible cylinder 40 is reduced. As such, distal ring 38 approaches proximal ring 36 axially along central axis CA. The approach of distal ring 38 to proximal ring 36 can be seen in the progress of FIGS. 6A-6E where the axial length between distal ring 38 and proximal ring 36 is reduced. The approach of distal ring 38 to proximal ring 36 is caused by a length of flexible cylinder 40 displacing radially inwards as distal ring 38 is twisted. FIGS. 6A-7 do not illustrate the deformation of flexible cylinder 40 in detail.

As distal ring 38 is twisted, curved protrusion 46 rotates with distal ring 38. As can be seen in FIGS. 6A-7, curved protrusion 46 follows locking path 76 as aperture valve 30 progresses through from first locking stage 66 to fifth locking stage 74. As best be seen in FIG. 6A, in first locking stage 66 curved protrusion 46 begins unrotated with respect to curved receptacle 44 relative central axis CA. FIGS. 6B-6E illustrate the movement of curved protrusion 46 relative to curved receptacle 44 as distal ring 38 rotates and translates axially relative to proximal ring 36 through successive snapshots. FIG. 6E illustrates curved protrusion 46 reversibly interlocking with curved receptacle 44. Curved protrusion 46 is held in curved receptacle 46 by the rotational restoring force of a elastically deformed flexible cylinder 40, as explained above.

Locking path 76 follows a tip of curved protrusion 46 along a helical trajectory from first locking stage 66 to fifth locking stage 74, illustrating an exaggerated path of curved protrusion 46 relative to curved receptacle 44. As illustrated in FIG. 7, locking path 76 rotationally overshooting the destination of curved receptacle 44 before the locking members interconnect. In the most general case, flexible deformation of flexible cylinder 40 can permit an overshoot or undershoot relative to central axis CA sufficient to seal on an insertable catheter device, with the particular degree of angular overshoot or undershoot determined by material and dimensional properties of the catheter device. The overshoot of curved protrusion 46 past curved receptacle 44 allows for the protrusion at the tip of curved protrusion 46 to interlock with curved receptacle 44. Although for explanatory purposes FIGS. 6A-7 illustrate locking path 76 as fully circumscribing flexible cylinder 40 (i.e. a rotation of >360 degrees), any degree of angular rotation can be used, so long as rotation is sufficient to form a fluidly sealing iris and generate a rotational bias capable of retaining curved protrusion 46 in engagement with curved receptacle 44. Although FIGS. 6A-7 are primarily described herein as employing curved receptacle 44 and curved protrusion 46, FIGS. 6A-7 can alternatively employ flat receptacle 62 and flat protrusion 64, or any similar latching mechanism facilitated by the rotational bias provided by twisting of flexible cylinder 40.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

An aperture valve for maintaining hemostasis, the aperture valve extending from a proximal end to a distal end along a primary axis and comprising: a proximal ring situated at the proximal end and formed of rigid material; a distal ring situated at the distal end and formed of rigid material; a flexible cylinder surrounding the primary axis, attached to and axially overlapping with the second ring section and the fourth ring section, wherein the flexible cylinder is irisable by rotation of the proximal ring relative to the distal ring; and a locking mechanism selectively anchoring the proximal ring to the distal ring, the locking mechanism engageable to retain the flexible cylinder in an irised state by locking the proximal ring to the distal ring while the proximal ring is rotated relative to the distal ring.

The aperture valve of the preceding paragraph can optionally include, additionally and/or alternatively, any one or more of the following features, configurations and/or additional components:

A further embodiment of the foregoing aperture valve, wherein the locking mechanism comprises: a protrusion extending from either the distal ring or the proximal ring axially towards the other of the distal ring and the proximal ring; and a receptacle disposed on the other of the distal ring and the proximal ring, the receptacle being configured to receive the protrusion.

A further embodiment of any of the foregoing aperture valves, wherein the protrusion is disposed radially outward of the flexible cylinder.

A further embodiment of any of the foregoing aperture valves, wherein the protrusion extends from the proximal ring and the receptacle is disposed on the distal ring.

A further embodiment of any of the foregoing aperture valves, wherein irising of the flexible cylinder from an undeformed state to a sealing irised state corresponds to rotation of the distal ring relative to the proximal ring through an irising angle relative to the primary axis.

A further embodiment of any of the foregoing aperture valves, wherein irising of the flexible cylinder from the undeformed state to the sealing irised state shortens an axial length of the flexible cylinder to a contracted axial length.

A further embodiment of any of the foregoing aperture valves, wherein the protrusion is angularly offset from the receptacle by the irising angle, and the protrusion extends the contracted axial length, such that rotation of the distal ring relative to the proximal ring from placing the flexible cylinder in the sealing irised state angularly and axially shifts the protrusion into alignment and engagement with the receptacle.

A further embodiment of any of the foregoing aperture valves, wherein the flexible cylinder is rotatable through an angle greater than the irising angle, such that twisting of the flexible cylinder generates a counterrotational restoring force retaining the protrusion in the receptacle.

A further embodiment of any of the foregoing aperture valves, wherein the flexible cylinder is formed of an elastically deformable material with elasticity sufficient to generate the counterrotational restoring force.

A further embodiment of any of the foregoing aperture valves, wherein the flexible cylinder is formed of a material selected from the group consisting of silicone, polyvinylchloride, polyethylene, polypropylene, polytetrafluoroethylene, polymethylmethacrylate, trimethyl carbonate, and combinations thereof.

A further embodiment of any of the foregoing aperture valves, wherein: the proximal ring comprises: a first ring section having a first outer diameter; and a second ring section having a second outer diameter less than the first outer diameter; the distal ring comprises: a third ring section having a third outer diameter; and a fourth ring section having a fourth outer diameter less than the third outer diameter; the aperture valve further comprises a plurality of teeth extending radially with respect to the primary axis from at least one of the group consisting of the third ring section and the fourth ring section; and the protrusion is disposed on either the first ring section or the third ring section, and the receptacle is disposed on the other of the first ring section and the third ring section.

A further embodiment of any of the foregoing aperture valves, wherein the receptacle extends radially outward from the other of the first ring section and the third ring section to receive the protrusion.

A further embodiment of any of the foregoing aperture valves, wherein the teeth extend radially outward from the at least one of the group consisting of the second ring section and the fourth ring section.

A further embodiment of any of the foregoing aperture valves, wherein the teeth extend radially from both the second ring section and the fourth ring section.

A further embodiment of any of the foregoing aperture valves, wherein the teeth extend radially outward from both the second ring section and the fourth ring section.

A further embodiment of any of the foregoing aperture valves, wherein the teeth are circumferentially distributed about the second ring section and the fourth ring section.

A further embodiment of any of the foregoing aperture valves, wherein radial engagement of the teeth with the flexible cylinder promotes formation of circumferentially overlapping irising folds in the flexible cylinder when the proximal ring is rotated relative to the distal ring.

A further embodiment of any of the foregoing aperture valves, wherein the flexible cylinder is attached to the second ring section and the fourth ring section via a melt process whereby the teeth melt into and bond with the flexible cylinder.

A further embodiment of any of the foregoing aperture valves, wherein irising of the flexible cylinder comprises deformation of the flexible cylinder into circumferentially overlapping irising folds when the proximal ring rotates with respect to the distal ring.

A further embodiment of any of the foregoing aperture valves, wherein an inner diameter of the flexible cylinder is reducible to a sealing diameter by the rotation of the proximal ring relative to the distal ring.

A further embodiment of any of the foregoing aperture valves, wherein the aperture valve is sized to receive an insertable medical device having an outer diameter less than a maximum of the inner diameter of the flexible cylinder, and substantially equal to the sealing diameter.

A further embodiment of any of the foregoing aperture valves, wherein the proximal ring further comprises a connecting ring section configured to be received on and form a fluid seal with a sealing stack of a guide sheath assembly.

A further embodiment of any of the foregoing aperture valves, wherein the connecting ring section comprises a tapered frustoconical portion at a most proximal end of the proximal ring, and a cap lock extending radially from proximal ring.

A further embodiment of any of the foregoing aperture valves, wherein the cap lock extends radially outward from the proximal ring.

A further embodiment of any of the foregoing aperture valves, wherein the tapered frustoconical portion of the connecting ring section defines an innermost diameter of the proximal ring, relative to the primary axis.

A further embodiment of any of the foregoing aperture valves, wherein a minimum inner diameter of the flexible cylinder in a sealing irised state is less than the innermost diameter of the proximal ring.

A further embodiment of any of the foregoing aperture valves, wherein the flexible cylinder is attached to the second ring section and the fourth ring section via adhesive.

A further embodiment of any of the foregoing aperture valves, wherein the flexible cylinder is attached to proximal and distal rings in a fluid-impermeable connection.

A further embodiment of any of the foregoing aperture valves, wherein the distal and proximal rings have a common inner diameter in a region abutting the flexible cylinder.

A further embodiment of any of the foregoing aperture valves, wherein the distal and proximal rings are disposed at least partially coaxially within the flexible cylinder.

A further embodiment of any of the foregoing aperture valves, wherein the proximal and distal rings have maximum outer diameters greater than an outer diameter of the flexible cylinder, and minimum outer diameters inboard of the flexible cylinder, wherein the maximum outer diameters transfer to the minimum inner diameters via steps abutting opposite ends of the flexible cylinder.

A further embodiment of any of the foregoing aperture valves, wherein the aperture valve is sterilized.

A further embodiment of any of the foregoing aperture valves, wherein the locking mechanism is a ratchetable fastener securable at multiple angular positions.

A guide sheath assembly for maintaining hemostasis during vascular insertion of an insertable medical device into a patient, the guide sheath assembly extending along a central axis from a proximal end to a distal end closest to the patient, the guide sheath assembly comprising: a sheath shaft coaxial with the central axis and disposed at the proximal end to axially receive the insertable medical device; and a seal stack forming a hemostatic seal distal from the sheath shaft, the seal stack comprising: a pre-insertion valve configured to form a hemostatic seal when the insertable medical device is not inserted through the guide sheath assembly; and an irising aperture valve coaxial with the central axis and located distally relative to the pre-insertion valve, the irising aperture valve comprising: a rigid proximal ring; a rigid distal ring axially translatable and angularly rotatable relative to the rigid proximal ring; and a flexible cylinder connecting the rigid proximal ring to the rigid distal ring and irisable with rotation of the rigid distal ring relative to the rigid proximal ring; and a handle providing an exterior housing retaining and at least partially coaxially surrounding the sheath shaft and the seal stack.

The guide sheath assembly of the preceding paragraph can optionally include, additionally and/or alternatively, any one or more of the following features, configurations and/or additional components:

A further embodiment of the foregoing guide sheath assembly, wherein the pre-insertion valve is a duckbill valve.

A further embodiment of any of the foregoing guide sheath assemblies, wherein the seal stack further comprises at least one insertion sealing valve disposed to form a hemostatic seal about the insertable medical device.

A further embodiment of any of the foregoing guide sheath assemblies, wherein the at least one insertion sealing valve comprises a fluid seal sized and shaped to receive a catheter.

A further embodiment of any of the foregoing guide sheath assemblies, wherein the at least one insertion sealing valve comprises a disc valve.

A further embodiment of any of the foregoing guide sheath assemblies, wherein the at least one insertion sealing valve comprises a fluid seal sized and shaped to receive a guide wire.

A further embodiment of any of the foregoing guide sheath assemblies, wherein the at least one insertion sealing valve comprises a cross slit valve.

A further embodiment of any of the foregoing guide sheath assemblies, further comprising a flush tube disposed at a location along the central axis between the seal stack and the sheath shaft.

A further embodiment of any of the foregoing guide sheath assemblies, wherein the pre-insertion valve is the most proximal element of the seal stack.

A further embodiment of any of the foregoing guide sheath assemblies, wherein the irising aperture valve is the most distal element of the seal stack.

A further embodiment of any of the foregoing guide sheath assemblies, wherein the handle coaxially surrounds only a proximal end of the irising aperture valve, such that the rigid distal ring of the irising axial valve extends distally away from and outside of the handle.

A further embodiment of any of the foregoing guide sheath assemblies, wherein the handle coaxially surrounds only a connecting ring section disposed at most proximal end of the rigid proximal ring of the irising aperture valve.

A further embodiment of any of the foregoing guide sheath assemblies, wherein the connecting ring section comprises a frustoconically tapered end with a narrower inner and outer diameter than the remainder of the rigid proximal ring.

A further embodiment of any of the foregoing guide sheath assemblies, wherein the connecting ring section a cap lock mechanically engaging the handle.

A further embodiment of any of the foregoing guide sheath assemblies, wherein the flexible cylinder is formed of an elastically deformable material that forms overlapping irising folds when the rigid distal ring is rotated relative to the rigid proximal ring.

A further embodiment of any of the foregoing guide sheath assemblies, wherein the irising aperture valve further comprises a plurality of teeth disposed at an intersection of the flexible cylinder and at least one of the rigid proximal and distal rings, such that the teeth extend into the flexible cylinder to promote the formation of the overlapping irising folds.

A further embodiment of any of the foregoing guide sheath assemblies, wherein the flexible cylinder overlaps axially with both the rigid proximal ring and the rigid distal ring.

A further embodiment of any of the foregoing guide sheath assemblies, wherein: the rigid distal ring comprises: a first ring section; and a second ring section proximally adjacent the first ring section; the rigid proximal ring comprises: a third ring section; and a fourth ring section distally adjacent the third ring section; the second and fourth ring sections at least partially axially overlap with the flexible cylinder; and a radial thickness of the second and fourth ring sections is less than a corresponding radial thickness of the first and third ring sections, respectively.

A further embodiment of any of the foregoing guide sheath assemblies, wherein the flexible cylinder is disposed radially outward of and at least partially coaxially overlapping with the second and fourth ring sections.

A further embodiment of any of the foregoing guide sheath assemblies, wherein the second and fourth ring sections have outer diameters less than the first and third ring sections, thereby forming annular steps abutting the flexible cylinder.

A further embodiment of any of the foregoing guide sheath assemblies, wherein the first and third ring sections have outer diameters greater than a maximum outer diameter of the flexible cylinder.

A further embodiment of any of the foregoing guide sheath assemblies, further comprising a plurality of teeth extending radially from and are distributed circumferentially about both the second ring section and the fourth ring section.

A further embodiment of any of the foregoing guide sheath assemblies, wherein irising of the flexible cylinder reduces an inner diameter of the flexible cylinder to a minimum diameter less than inner diameters of the rigid proximal ring and the rigid distal ring.

A further embodiment of any of the foregoing guide sheath assemblies, wherein the flexible cylinder is attached to the rigid proximal ring and the rigid distal ring in a fluid seal by adhesive.

A further embodiment of any of the foregoing guide sheath assemblies, wherein the flexible cylinder is attached to the rigid proximal ring and the rigid distal ring in a fluid seal by a melt process whereby the teeth melt into and bond with the flexible cylinder.

A further embodiment of any of the foregoing guide sheath assemblies, wherein the aperture valve is sterilized.

A further embodiment of any of the foregoing guide sheath assemblies, wherein irising of the flexible cylinder comprises deformation of the flexible cylinder into circumferentially overlapping irising folds when the proximal ring rotates with respect to the distal ring.

A further embodiment of any of the foregoing guide sheath assemblies, further comprising a locking mechanism securing the rigid proximal ring to the rigid distal ring to prevent rotation when the flexible cylinder is in an irised state.

A further embodiment of any of the foregoing guide sheath assemblies, wherein the locking mechanism comprises: a receptacle disposed on one of the rigid proximal ring and the rigid distal ring; and a protrusion disposed on the other of the rigid proximal ring and the rigid distal ring and shaped to latch into the receptacle.

A further embodiment of any of the foregoing guide sheath assemblies, wherein counterrotational bias of the flexible cylinder, when in the irised state, retains the protrusion in the receptacle.

A method of forming a hemostatic seal about a medical device at a vascular insertion site, the method comprising: extending the medical device through a sheath shaft to the insertion site, along a central axis; interposing an aperture seal valve fluidly sealed with the sheath shaft between the sheath shaft and the insertion site, the aperture seal valve comprising: a proximal ring; a distal ring axially translatable and angularly rotatable relative to the rigid proximal ring; a flexible cylinder connecting the proximal ring to the distal ring and irisable by rotation of the distal ring relative to the proximal ring; a plurality of teeth disposed at an intersection of the flexible cylinder and at least one of the proximal and distal rings; and a locking mechanism; rotating the distal ring relative to the proximal ring, thereby irising the flexible cylinder to form a fluid seal about the medical device; and engaging the locking mechanism to lock the proximal ring relative to the distal ring, thereby retaining the fluid seal about the medical device.

The method of the preceding paragraph can optionally include, additionally and/or alternatively, any one or more of the following features, configurations and/or additional steps:

A further embodiment of the foregoing method, wherein the locking mechanism comprises: a protrusion extending axially from one of the proximal ring and the distal ring toward the other of the proximal ring and the distal ring; and a receptacle disposed on the other of the proximal ring and the distal ring.

A further embodiment of any of the foregoing methods, wherein engaging the locking mechanism comprises latching the protrusion to the receptacle.

A further embodiment of any of the foregoing methods, wherein rotating the distal ring relative to the proximal ring moves the protrusion through a helical locking path, relative to the central axis.

A further embodiment of any of the foregoing methods, wherein the locking path shortens an axial distance between the distal ring and the proximal ring, and traverses an angular displacement corresponding to the irising of the flexible cylinder.

A further embodiment of any of the foregoing methods, wherein the locking path terminates at an angular displacement sufficient to iris the flexible cylinder to form the fluid seal about the medical device.

A further embodiment of any of the foregoing methods, wherein the locking path overshoots the angular displacement sufficient to iris the flexible cylinder to form the fluid seal about the medical device, then backs into engagement of the locking mechanism, such that a counterrotational bias of the irised flexible cylinder retains the protrusion in the receptacle.

A further embodiment of any of the foregoing methods, further comprising forming a second hemostatic seal between the sheath shaft and the aperture seal valve.

A further embodiment of any of the foregoing methods, wherein the second hemostatic seal is provided by a disk valve.

A further embodiment of any of the foregoing methods, wherein the second hemostatic seal is provided by a cross slit valve.

A further embodiment of any of the foregoing methods, wherein the irising of the flexible cylinder is promoted by engagement of the plurality of teeth with the flexible cylinder.

The above method(s) can be performed on a living animal or on a simulation, such as on a cadaver, cadaver heart, anthropomorphic ghost, simulator (e.g., with body parts, heart, tissue, etc. being simulated).

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. An aperture valve for maintaining hemostasis, the aperture valve extending from a proximal end to a distal end along a primary axis and comprising:
a proximal ring situated at the proximal end and formed of rigid material;
a distal ring situated at the distal end and formed of rigid material;
a flexible cylinder formed of elastically deformable material and surrounding the primary axis, attached to and axially overlapping with the proximal ring and the distal ring, wherein the flexible cylinder is irisable by rotation of the proximal ring relative to the distal ring;
a plurality of teeth extending radially with respect to the primary axis that engage the flexible cylinder to promote folding of the flexible cylinder into an irised shape with overlapping folds to reduce an inner diameter of the flexible cylinder; and
a locking mechanism selectively anchoring the proximal ring to the distal ring, the locking mechanism engageable to retain the flexible cylinder in an irised state by locking the proximal ring to the distal ring while the proximal ring is rotated relative to the distal ring.

2. The aperture valve of claim 1, wherein the locking mechanism comprises:
a protrusion extending from either the distal ring or the proximal ring axially towards the other of the distal ring and the proximal ring; and
a receptacle disposed on the other of the distal ring and the proximal ring, the receptacle being configured to receive the protrusion.

3. The aperture valve of claim 2, wherein the protrusion is disposed radially outward of the flexible cylinder.

4. The aperture valve of claim 2, wherein irising of the flexible cylinder from an undeformed state to a sealing irised state corresponds to rotation of the distal ring relative to the proximal ring through an irising angle relative to the primary axis.

5. The aperture valve of claim 2, wherein:
the proximal ring comprises:
a first ring section having a first outer diameter; and
a second ring section having a second outer diameter less than the first outer diameter;
the distal ring comprises:
a third ring section having a third outer diameter; and
a fourth ring section having a fourth outer diameter less than the third outer diameter;
the plurality of teeth extend radially with respect to the primary axis from at least one of the group consisting of the third ring section and the fourth ring section; and
the protrusion is disposed on either the first ring section or the third ring section, and the receptacle is disposed on the other of the first ring section and the third ring section.

6. The aperture valve of claim 5, wherein the receptacle extends radially outward from the other of the first ring section and the third ring section to receive the protrusion.

7. The aperture valve of claim 5, wherein the teeth extend radially outward from the at least one of the group consisting of the second ring section and the fourth ring section.

8. The aperture valve of claim 1, wherein irising of the flexible cylinder comprises deformation of the flexible cylinder into circumferentially overlapping irising folds when the proximal ring rotates with respect to the distal ring.

9. The aperture valve of claim 8, wherein an inner diameter of the flexible cylinder is reducible to a sealing diameter by the rotation of the proximal ring relative to the distal ring.

10. The aperture valve of claim 1, wherein the flexible cylinder is attached to proximal and distal rings in a fluid-impermeable connection.

11. The aperture valve of claim 1, wherein the locking mechanism is a ratchetable fastener securable at multiple angular positions.

12. A guide sheath assembly for maintaining hemostasis during vascular insertion of an insertable medical device into a patient, the guide sheath assembly extending along a central axis from a proximal end to a distal end closest to the patient, the guide sheath assembly comprising:
a sheath shaft coaxial with the central axis and disposed at the proximal end to axially receive the insertable medical device; and
a seal stack forming a hemostatic seal distal from the sheath shaft, the seal stack comprising:
a pre-insertion valve configured to form a hemostatic seal when the insertable medical device is not inserted through the guide sheath assembly; and
an irising aperture valve coaxial with the central axis and located distally relative to the pre-insertion valve, the irising aperture valve comprising:
a rigid proximal ring;
a rigid distal ring axially translatable and angularly rotatable relative to the rigid proximal ring; and
a flexible cylinder formed of elastically deformable material and connecting the rigid proximal ring to the rigid distal ring and irisable with rotation of the rigid distal ring relative to the rigid proximal ring; and
a plurality of teeth extending radially with respect to the central axis that engage the flexible cylinder to promote folding of the flexible cylinder into an irised shape with overlapping folds to reduce an inner diameter of the flexible cylinder; and
a handle providing an exterior housing retaining and at least partially coaxially surrounding the sheath shaft and the seal stack.

13. The guide sheath assembly of claim 12, further comprising a flush tube disposed at a location along the central axis between the seal stack and the sheath shaft.

14. The guide sheath assembly of claim 12, wherein the irising aperture valve is the most distal element of the seal stack.

15. The guide sheath assembly of claim 14, wherein the handle coaxially surrounds only a proximal end of the irising aperture valve, such that the rigid distal ring of the irising axial valve extends distally away from and outside of the handle.

16. The guide sheath assembly of claim 12, wherein irising of the flexible cylinder comprises deformation of the flexible cylinder into circumferentially overlapping irising folds when the proximal ring rotates with respect to the distal ring.

17. The guide sheath assembly of claim 12, further comprising a locking mechanism securing the rigid proximal ring to the rigid distal ring to prevent rotation when the flexible cylinder is in an irised state.

18. The guide sheath assembly of claim 17, wherein the locking mechanism comprises:
a receptacle disposed on one of the rigid proximal ring and the rigid distal ring; and
a protrusion disposed on the other of the rigid proximal ring and the rigid distal ring and shaped to latch into the receptacle.

19. The guide sheath assembly of claim 18, wherein counterrotational bias of the flexible cylinder, when in the irised state, retains the protrusion in the receptacle.

20. A seal stack for maintaining hemostasis, the seal stack comprising:
a pre-insertion valve configured to form a hemostatic seal when an insertable medical device is not inserted through the seal stack; and
an irising aperture valve located distally relative to the pre-insertion valve, the irising aperture valve comprising:
a rigid proximal ring;
a rigid distal ring axially translatable and angularly rotatable relative to the rigid proximal ring;
a flexible cylinder attached to and axially overlapping with the ridged proximal ring and the rigid distal ring, wherein the flexible cylinder is irisable by rotation of the rigid proximal ring relative to the rigid distal ring;
a plurality of radial protrusions that engage the flexible cylinder to promote folding of the flexible cylinder into an irised shape with overlapping folds to reduce an inner diameter of the flexible cylinder; and
a locking mechanism selectively anchoring the proximal ring to the distal ring.
